(19) United States
(12) Patent Application Publication
(10) Pub. No.: US 2024/0183847 A1
(43) Pub. Date: Jun. 6, 2024

(54) IMPEDANCE-BASED NEUTRALIZING ANTIBODY DETECTION BIOSENSOR WITH APPLICATION TO SARS-CoV-2 INFECTION

(71) Applicant: Southern Methodist University, Dallas, TX (US)

(72) Inventors: Jungchih Chiao, Grand Prairie, TX (US); Ali Beskok, Plano, TX (US)

(21) Appl. No.: 18/522,587

(22) Filed: Nov. 29, 2023

Related U.S. Application Data
(60) Provisional application No. 63/385,706, filed on Dec. 1, 2022.

(51) Int. Cl. G01N 33/543; G01N 27/22; G01N 33/569

(52) U.S. Cl. CPC G01N 33/5438; G01N 27/227; G01N 33/56983; G01N 2333/165; G01N 2469/20

(57) ABSTRACT

A neutralizing antibody detection system comprising a plurality of electrodes, wherein neutralizing antibody antigens are bound to a respective surface of each electrode, and wherein the plurality of electrodes with the neutralizing antibody antigens are configured to bind virions when immersed in a solution containing the virions; and an impedance analyzer electrically connected to the plurality of electrodes, wherein the impedance analyzer is configured to apply a voltage at a specified frequency and to measure a respective impedance at the specified frequency.

### CROSS-REFERENCE TO RELATED APPLICATIONS

This application claims priority to U.S. Provisional Application Ser. No. 63/385,706, filed Dec. 1, 2022, the entire contents of which is incorporated herein by reference.

### STATEMENT OF FEDERALLY-FUNDED RESEARCH

Not applicable.

### TECHNICAL FIELD OF THE INVENTION

The present invention relates in general to antibody detection and, more particularly, to impedance-based neutralizing antibody detection.

### BACKGROUND OF THE INVENTION

Although safe and efficacious coronavirus disease-2019 (COVID-19) vaccines are available, real protective immunity is revealed by the serum COVID-19 neutralizing antibody (NAb) concentration. Neutralizing antibodies deactivate the virus by attaching to the viral receptor-binding domain (RBD), which interacts with angiotensin-converting enzyme 2 (ACE2) on the human cell. The present invention introduces inexpensive, rapid, sensitive, and quantifiable impedance-based immunosensors to evaluate the NAb. Sensor limit of detection (LOD) is experimentally determined in different buffer dilutions using bovine IgG-anti-bovine IgG interaction. The dominance of AC electrokinetics phenomena and molecular diffusion in the sensor is investigated using scaling analysis and numerical simulations. The results demonstrated that the sensor detection mechanism is mainly based on the diffusion of the biomolecules onto the electrode surface. After evaluating the sensor working principles, viral RBDs buffers, including different NAb concentrations, are applied to the sensor, immobilized with the human ACE2 (hACE2). Results demonstrate the sensor is capable of NAb detection in the analytical measuring interval (AMI) between 45 (ng/ml) to 185 (ng/ml). Since the present sensor provides fast test results with lower costs, it can be used to assess NAb in people's blood serum before receiving further COVID vaccine doses.

Coronavirus disease-2019 (COVID-19) is an unprecedented pandemic. COVID-19 originated from severe acute respiratory syndrome coronavirus 2 (SARS-CoV-2). The first step of SARS-CoV-2 entry into host cells is the binding of the spike (S) protein via receptor-binding domain (RBD) to angiotensin-converting enzyme 2 (ACE2) and subsequent membrane fusion [1]. After SARS-CoV-2 infection or vaccination, serum-neutralizing antibodies (NAbs) rapidly appear to inactivate the viral RBDs [2]. However, the concentrations of NAbs obtained by current vaccines have been shown to vary by as much as 25-fold after several months [3]. The individualized evaluation of the neutralizing capacity of anti-SARS-CoV-2 antibodies is important because it can provide insights into real protective immunity. However, continuous testing of a large population requires the development of accurate, fast, quantifiable, and inexpensive biosensors based on detecting human antibodies.

While antibody and antigen detections with lateral flow assays are available [4], which are quick, affordable, and accessible without tedious sample processing logistics issues, they do not provide quantitative values to assess antibodies. Currently, the lateral flow technology is not capable of neutralizing antibody assessment either. Standard virus plaque reduction neutralization test assays used to evaluate NAbs in blocking live infectious SARS-CoV-2 virus require their operation in high biosafety rating laboratories [5]. The procedures are time-consuming, expensive, and require skilled operators. GenScript's cPass neutralization antibody assay has received Emergency Use Authorization (EUA) use by U.S. Food and Drug Administration (FDA) [6]. However, the detection kit provides semi-quantitative evaluation and requires skills to operate. Several methods have been proposed to pursue affordable and convenient point-of-care NAb detection methods. A vertical-flow cellulose paper-based assay showed comparable detection results to lab-based ones [7]. The method measures the interaction between RBD and ACE2 receptors, and the high-affinity signals specific to NAbs enable fast evaluation without the requirement of live viruses. A workflow for cellulose pull-down virus neutralization tests (cpVNT) and optical image acquisition for colorimetric signal processing are needed for further process refinement, automation, and electronics integration for the vertical flow assays [7-9]. In comparison, electrochemical sensors also show great promise as they provide high sensitivity, shorter detection time, accurate selectivity, and lower costs [10].

In this type of sensor, electrodes are the main part where biomolecules (e.g., enzyme [11], antibody [12], and nucleic acid [13]) are immobilized via different approaches [14]. Then, antigen-antibody interactions (affinity biosensors) on the electrode surface are transduced to electrical signals (e.g., impedance, current, voltage, etc.) [15]. Detection in affinity biosensors is based on specific binding between determined reagents (e.g., a receptor, nucleic acid, or an antibody). Therefore, the interaction of the biological component and the analyte is crucial to achieving detection [16]. The capacitive sensing method is one of the introduced alternating current electrokinetic (ACEK) based immunosensors [17]. Applying AC electric potentials in ionized media induces diffuse charge at the electrode/electrolyte interface or electric double layer (EDL). Molecular binding is characterized by changes in the EDL's electrical signal (capacitance change rate). Capacitive sensing allows real-time measurements of fast binding processes [17, 18] and has been used for various applications [18-21]. Alternating current electrokinetic (ACEK) transport phenomena, including AC Electroosmosis (ACEO), AC Electrothermal (ACET), and Dielectrophoresis (DEP), are widely reported to enhance detection in affinity biosensors in the references. However, there is no scaling analysis to prove these claims.

### SUMMARY OF THE INVENTION

An embodiment of the present invention includes a neutralizing antibody detection system including: a plurality of electrodes, wherein neutralizing antibody antigens are bound to a respective surface of each electrode, and wherein the plurality of electrodes with the neutralizing antibody antigens are configured to bind virions when immersed in a solution containing the virions; and an electrical circuit connected to the plurality of electrodes to apply a voltage at a specified frequency and to measure a respective impedance. In one aspect, the plurality of electrodes includes interdigitated electrodes. In another aspect, the plurality of electrodes includes a layer of conductive material. In another aspect, the virions are SARS-CoV-2 virions and the neutralizing antibody antigens are SARA-CoV-2 neutralizing antibody antigens. In another aspect, the specified frequency is greater than or equal to 10 Hz and less than or equal to 1000 Hz. In another aspect, the specified frequency is greater than or equal to 100 Hz and less than or equal to 1000 Hz. In another aspect, the specified frequency is greater than or equal to 100 Hz and less than or equal to 600 Hz. In another aspect, the specified frequency is 100 Hz, 200 Hz, 300 Hz, 400 Hz, 500 Hz, 600 Hz, 700 Hz, 800 Hz, 900 Hz, or 1000 Hz.

Another embodiment of the present invention includes a method of detecting neutralizing antibodies including providing a plurality of electrodes; providing a plurality of electrodes; binding neutralizing antibody antigens to a respective surface of each electrode; immersing the plurality of electrodes in a solution that may include virions and neutralizing antibodies; applying a voltage at a specified frequency to the plurality of electrodes immersed in the solution; measuring an impedance; and calculating a concentration of neutralizing antibodies from the measured impedance. In one aspect, the plurality of electrodes includes interdigitated electrodes. In another aspect, the plurality of electrodes includes a layer of conductive material. In another aspect, the neutralizing antibody antigens are human angiotensin-converting enzyme 2 receptors. In another aspect, the virions are SARS-CoV-2 virions and the neutralizing antibody antigens are SARA-CoV-2 neutralizing antibody antigens. In another aspect, the specified frequency is greater than or equal to 10 Hz and less than or equal to 1000 Hz. In another aspect, the specified frequency is greater than or equal to 100 Hz and less than or equal to 1000 Hz. In another aspect, the specified frequency is greater than or equal to 100 Hz and less than or equal to 600 Hz. In another aspect, the specified frequency is 100 Hz, 200 Hz, 300 Hz, 400 Hz, 500 Hz, 600 Hz, 700 Hz, 800 Hz, 900 Hz, or 1000 Hz.

### BRIEF DESCRIPTION OF THE DRAWINGS

For a more complete understanding of the features and advantages of the present invention, reference is now made to the detailed description of the invention along with the accompanying figures and in which:

FIG. 1A shows a commercially available SAW resonator, FIG. 1B shows a modification of the SAW resonator, and FIG. 1C shows a schematic for the experimental setup.

FIG. 2 shows RBDs that are neutralized by the NAbs not interacting with hACE2 receptors on the electrode surfaces, while the non-neutralized RBDs link to the hACE2 receptors.

FIGS. 3A and 3B show voltage and antibody immobilization effects on impedance magnitude (FIG. 3A) and phase angle of the chip impedance (FIG. 3B).

FIGS. 3C and 3D show impedance magnitudes before (t=0) and after (t=120 s) antibody-antigen interaction at different PBS concentrations (FIG. 3C) and impedance magnitude changes before and after antibody-antigen interaction in the frequency range from 100 Hz to 10 MHz for different PBS dilutions (FIG. 3D).

FIGS. 4A, 4B, 4C, and 4D show detection of different antigen concentrations in A) 0.01×PBS buffer (FIG. 4A), 0.1×PBS buffer (FIG. 4B), and 1×PBS buffer (FIG. 4C). and the standard deviation of LOD for each PBS buffer (FIG. 4D), where all data for 0.01×PBS and 0.1×PBS were obtained at 100 mV and 100 Hz, and for 1×PBS, the data was obtained at 100 mV and 600 Hz.

FIGS. 5A and 5B show concentration results from simulations. FIG. 5A shows a contour plot of the normalized anti-bovine IgG concentration ([A]/[A₀]) at the initial time (t=0) and (t=120 s). FIG. 5B shows a normalized concentration of the interacted anti-bovine IgG-bovine IgG ([AB]/[A₀]) on the surface of the electrodes.

FIG. 6A shows the LOD for RBD-hACE2 interaction for different NAbs concentrations, and FIG. 6B shows the NAb concentration and impedance change relationship for the device, where all data were obtained using 0.1×PBS buffer for dilution at 100 mV and 100 Hz with the initial impedance magnitude of 100 kΩ.

FIG. 7A shows ACE2 immobilization effects on impedance magnitude. FIG. 7B shows the phase angle of the chip impedance. FIG. 7C shows the LOD for RBD-hACE2 interaction for different NAbs concentrations. FIG. 7D shows the NAb concentration and impedance change relationship for the device.

FIG. 8 shows a flowchart for a method embodiment of the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The terminology used and specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

In this work, an interdigitated electrode was modified as a biosensor to characterize SARS-CoV-2 NAbs. The interaction between bovine IgG and anti-bovine IgG characterized the sensor's detection potential. A wide range of frequencies varying from 100 Hz to 10 MHZ and different voltage magnitudes were considered to find the optimum detection frequency and voltage. The impedance spectroscopy technique was chosen to record the impedance of the tested devices in real time during antibody-antigen interaction on the electrode surface. Then, a scaling analysis was used to find the order of magnitude of the ACEK forces, including ACEO, ACET, and DEP, in three different PBS buffer dilutions (0.01×PBS, 0.1×PBS, and 1×PBS). The orders of magnitude of antibody displacement due to these forces were compared with the Brownian motion of the IgGs, which causes diffusion. A numerical simulation technique was employed to demonstrate the validity of the theoretical analysis. Then, the device was employed for SARS-CoV-2 NAbs detection. When a person gets affected by SARS-CoV-2, viral RBDs attach to the human ACE2 (hACE2) and enter the host cell through membrane fusion. Viral RBDs are neutralized by NAbs and thus deactivated. The NAbs concentration in the blood serum determines immunity strength after vaccination. To evaluate this concentration using the proposed device, the electrode surface was immobilized with human ACE2 (hACE2), reacting to the viral RBD. Then, the RBD buffers with different NAb concentrations were applied to the device, and the resultant electrical signals were recorded.

This study utilized the impedance spectroscopy technique to evaluate antibody-antigen binding on the electrode interfaces. The method applies an alternative current (AC) electric field as the stimulus and the corresponding electrical current through the system as the response is measured. Since the applied voltage is frequency dependent, a range of frequencies is applied to obtain comprehensive information from the system to produce an impedance spectrum. Therefore, any change in the impedance spectrum can be interpreted as an electrode/electrolyte interfacial change. Parameters such as electrode potential, temperature, ion concentration, an oxide layer, electrode surface roughness, and impurity adsorption may also result in an interfacial change. However, the sensors were washed and plasma cleaned before any further step, the experiments were performed in a temperature-controlled environment, and purified buffers were used for dilution purposes. Therefore, the impedance changes were interpreted as molecular bindings. These changes can be described using a simplified equivalent circuit model. EDL and the solution are considered an interfacial capacitor, Cᵢₙₜ on the electrodes, and buffer resistance, Rₛ, so the device impedance is approximated as Z=Rₛ+1/jωCᵢₙₜ or Z=|Z|e^{iΘ}. Molecular bindings increase the EDL thickness, which will change the interfacial impedance. Therefore, these changes are considered the binding index.

In previous studies, ACEK phenomena have been suggested to enhance detection by increasing mixing time. ACEO, ACET, and DEP are the main ACEK phenomena that induce forces on the antibodies and bring them to the detection region [17-19]. It is important to understand the relative effects of ACEO, ACET, and DEP forces on sensor performance. For example, the suspended particles can experience negative DEP at certain frequencies depending on the ionic conductivity, which will repel them from the electrode surfaces [22]. Also, the ACET flow velocity varies with the 4^{th} power of the applied electric potential [23]. Therefore, high electric potentials can create large temperature variations that can denature proteins on the sensor surface, while increased fluid velocities can lead to large shear stresses on the sensor surfaces. However, positive DEP will enhance detection by attracting proteins and particles onto the sensor surfaces, and mild ACET flows can enhance mixing and enable convection for fast measurements.

Here we consider the relative magnitudes of the resultant displacement due to these forces during detection using several theoretical models. The magnitudes of these displacements are compared with the displacement due to Brownian motion. These models are used to determine the dominancy of each phenomenon for different PBS dilutions. Details for these models are provided in the literature [23-25]. The ACEO flow is generated in low conductive fluid due to the electric field effects on induced diffuse charges near the electrode surface or EDL. The specimen displacement in the buffer due to the ACEO flow from scaling analysis is [24]:

$${X}_{A C E O} = Λ \frac{ε {V}^{2}}{8 η r} \frac{{Ω}^{2}}{{( 1 + {Ω}^{2} )}^{2}} t \qquad ( 1 )$$

where ε is the solution permittivity (7.083×10⁻¹⁰ F/m), η is the dynamic viscosity of the solution (0.001 Pa·s), r is the half of the electrode spacing (1 μm), V is the applied voltage, Λ defined as Cₛ/(Cₛ+C_{D}), where Cₛ is the capacitance of the Stern layer (0.007 F/m²) [25], C_{D}=ε/λ_{D} is the capacitance of the diffuse layer, and Ω is equal to Λωεπr/2σλ_{D}, where ω is the radian frequency (2πf), t is time, σ is the conductivity of the fluid as 0.016 (S/m), 0.16 (S/m), 1.6 (S/m) for 0.01×PBS, 0.1×PBS, 1×PBS, respectively, and λ_{D} is the Debye length which is 0.7 (nm), 2.4 (nm), 7.4 (nm) for 1×PBS, 0.1×PBS, 0.01×PBS, respectively [26].
ACET arises from Joule heating due to the electric current passing through the buffer increasing with increased ionic conductivity and inducing ACET flow in the fluid due to the temperature dependencies of the electrical permittivity and conductivity of the medium [27, 28]. The ACET displacement of the particle is also found from scaling analysis as [23]:

$${X}_{A C E T} = \frac{1}{192 {π}^{2}} \frac{M}{T} \frac{ε σ {V}^{4}}{k η {L}_{C}} t \qquad ( 2 )$$

where T is the temperature (293.15 K), k is the thermal conductivity (0.6 W/m·K), L_{c} is the characteristic length for electrothermal flow, which is equal to the typical dimension of the chip, and M is the electrothermal factor defined as:

$$M = \frac{{( {C}_{σ} - {C}_{ε} )}^{T}}{1 + {( \frac{ω ε}{σ} )}^{2}} + \frac{1}{2} {C}_{ε} T \qquad ( 3 )$$

In this equation,

$${{{C}_{σ} = \frac{1}{σ ( {T}_{0} )} \frac{\partial σ}{\partial T} \vert}_{{T}_{0}} \approx 0.02 {K}^{- 1} and {C}_{ε} = \frac{1}{ε ( {T}_{0} )} \frac{\partial ε}{\partial T} \vert}_{{T}_{0}} \approx - 0.0 \; 04 {K}^{- 1} .$$

A dielectrophoretic (DEP) force is imposed on the suspended particles based on their relative polarizability with respect to the ionic fluid [29]. At a given AC frequency, more polarizable particles than the fluid are attracted to the high electric field regions near the electrodes, exhibiting positive DEP [30]. Otherwise, they are repelled from the near-electrode regions (i.e., negative DEP). The induced displacement on the particles due to the DEP force can be found as [24]:

$${X}_{D E P} = \frac{1}{3 {π}^{2}} R e \{ K \} \frac{{α}^{2} ε}{η} \frac{{β}^{2} {V}_{0}^{2}}{{r}^{3}} t \qquad ( 4 )$$

where a is the particle radius

$$( 2 a = 10 nm [ 26 ] ) , β is {Ω}^{2} / ( 1 + {Ω}^{2} ) , K = [ \frac{{ε}_{p}^{*} - {ε}_{m}^{*}}{{ε}_{p}^{*} + 2 {ε}_{m}^{*}} ]$$

is the Clausius-Mossotti (CM) factor, and ε*ₚ and ε*ₘ are the complex permittivities of particle and medium, respectively. Complex permittivity is

$${ε}^{*} = ε - j ( \frac{σ}{ωε} ) with {ε}_{p} = 3.54168 \times {10}^{- 11} F / m , {σ}_{p} = 0.22 S / m [ 31 , 32 ] .$$

The induced displacement of the particles due to the Brownian motion can be found in [25]:

$${X}_{B r o w n i a n} = {( \frac{{k}_{B} T}{3 π a η} t )}^{\frac{1}{2}} \qquad ( 5 )$$

where k_{B} is Boltzmann constant (1.38E−23 (m²·kg/s²·K)) and T is temperature in K.

The antibody-antigen interaction on the sensor's electrode surface has also been simulated. The following equations are considered to model this binding procedure.

The binding of the protein pair is obtained from the following chemical reaction:

$${[ A ]}_{surface} + [ B ] {{↔}_{{k}_{off}}}^{{k}_{on}} [ AB ] \qquad ( 6 )$$

where [A]_{Surface} is the target molecule concentration, [B] is surface receptor concentration, and [AB] is protein-protein complex concentration. The kₒₙ is the association rate constant (2.5E5 (1/M·s)), and k_{off} is the dissociation rate constant (3E−4 (1/s)) for the IgG-anti-IgG binding interactions [33]. The surface reaction can be found from the following first-order Langmuir adsorption equation:

$$\frac{\partial [ A B ]}{\partial t} = {k}_{o n} [ A ] ( [ {B}_{0} ] - [ A B ] ) - {k}_{o f f} [ AB ] \qquad ( 7 )$$

In this equation, [B₀] is the initial surface concentration of the receptor (1.4×10⁻⁸ (mol/m²)) [33]. Fick's second law describes the transport of analytes around the surface as follows:

$$\frac{\partial [ A ]}{\partial t} - D {∇}^{2} [ A ] = R \qquad ( 8 )$$

where [A] is the bulk concentration of the target molecule and D is its diffusion coefficient (5×10⁻¹¹ (m²/s)) [33]. The initial concentration of [A] is assumed to be [A₀] in the medium. These equations are solved using the COMSOL Multiphysics.

### Experimental Setup

### Bovine Antibody and Antigen Detection Procedure

Buffers of 1×PBS (˜1.6 S/m), 0.1×PBS (˜0.16 S/m), and 0.01×PBS (˜0.016 S/m) were prepared by 1:10, 1:100, and 1:1000 volume dilution of physiological strength stock solution (10×PBS, Fisher Scientific) with ultrapure deionized (DI) water. To determine the LOD of the present sensor, goat anti-bovine IgG (H+L) antibody (Jackson ImmunoResearch Laboratories Inc.) binding to bovine IgG whole molecules (Jackson ImmunoResearch Laboratories Inc.) was investigated. The bovine IgG was diluted with proper PBS buffer to 10 μg/mL for immobilization on the electrode surface. Also, the anti-bovine IgG was diluted with PBS to concentrations from 1 ng/ml to 10,000 ng/ml based on the buffer conductivity for detection purposes.

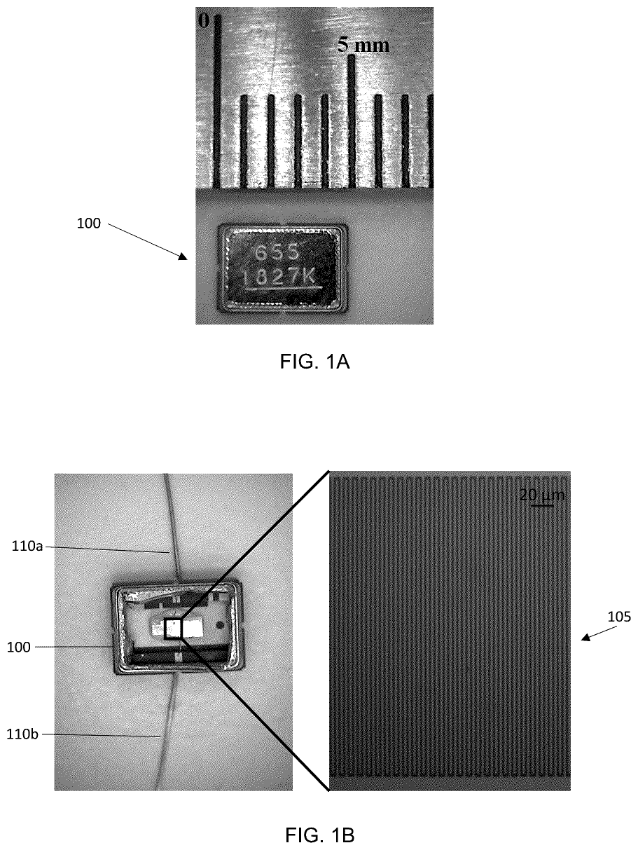

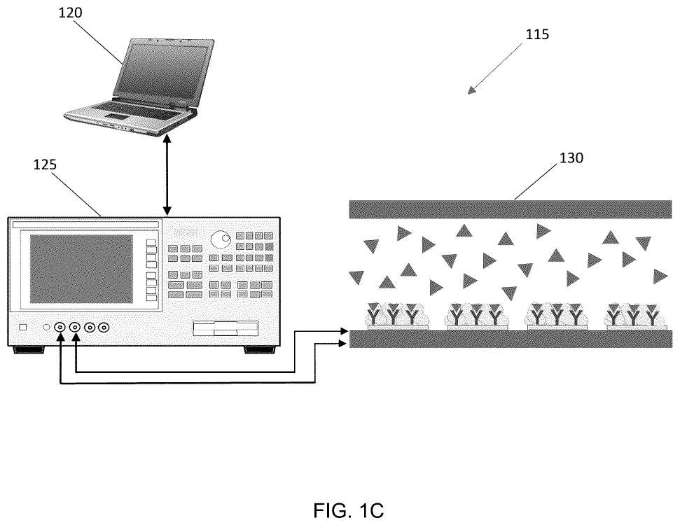

FIG. 1A shows a commercially available SAW resonator 100 (RO3101A, Murata Electronics) that was modified to employ the interdigitated microelectrode arrays for antibody-antigen binding purposes. This type of resonator has a small electrode size/spacing (2 μm/2 μm), made up of aluminum at a low price, making biosensing more affordable and accurate than other methods like ELISA. The modification includes removing the metallic cap on top of the electrodes 105 and connecting two wires 110a, 110b as shown in FIG. 1B. Then, the resonator was cleaned thoroughly using acetone, isopropyl alcohol, and DI water for 15 s for each. The electrodes 105 are placed inside a 5 mm×3.5 mm×1.5 mm container, which serves as a microchamber. In previous studies, an increase in the hydrophilicity of the surface using oxygen plasma cleaner was introduced to enhance the immobilization of the protein on the electrode surface [18]. Therefore, the electrode surface for different devices was treated with plasma cleaner (PDC-32G, HARRICK PLASMA) for 30 s. Then, IgG whole molecule buffer can wet the electrode surface properly. It should be noted that all the tested devices were plasma-treated 30 s before immobilization.

After plasma treatment, the chamber was filled with bovine IgG whole molecules (10 μg/ml) diluted with PBS solution. The loaded chip was kept inside a humidity chamber at 4° C. for 12 hours to functionalize the electrodes. Afterward, the unbounded IgG on the electrodes was washed away using PBS, and the chip was ready to detect anti-bovine IgG.

To start the detection, the chip wires were connected to a high-accuracy impedance analyzer (HP Agilent 4194A) which applied the desired voltage in the specified frequency and recorded the impedance simultaneously. FIG. 1C shows the schematic for the experimental setup 115, including a control computer 120, the impedance analyzer 125, and the detection mechanism 130. To assure the accuracy of the applied voltage and frequency by the impedance analyzer, an oscilloscope (TDS 2014B, Tektronix) was used to measure these two parameters before starting the experiments. Then, the chamber was filled with the diluted anti-bovine IgG in desired concentrations, and impedance changes were recorded continuously while the AC voltage was applied to the chip.

### Sensor Specificity and Selectivity Test

In order to study the specificity of the sensor, IgG-free bovine serum albumin (BSA) diluted with 0.1×PBS (1% BSA) was used as the control buffer and to dilute the anti-bovine IgG. In order to make 1% BSA, 1 g of IgG-free BSA was diluted in 100 mL of 0.1×PBS and well mixed. Then, the anti-bovine IgG was diluted with this buffer to 1 ng/ml and 10 ng/ml concentrations. For selectivity test purposes, 1:20 diluted mouse serum (Jackson ImmunoResearch Laboratories Inc.) with 0.1×PBS was used. The 1 ng/ml and 10 ng/ml anti-bovine IgG were diluted with mouse serum to test the sensor's selectivity. It should be noted that the supplied bovine IgG had minimal cross-reactivity with mouse serum proteins.

### SARS-CoV-2 Nab Detection Procedure

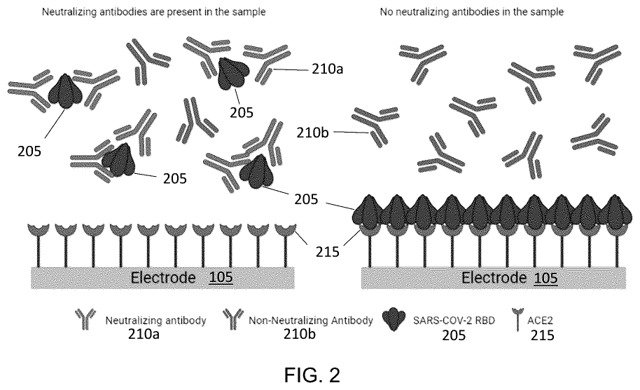

When the SARS-CoV-2 RBDs 205 are introduced into the biosensors (exemplars of which are indicated in FIG. 2), the RBDs that are neutralized by the NAbs 210a (exemplars of which are indicated in FIG. 2, with exemplar non-neutralizing antibodies 210b) will not interact with hACE2 receptors 215 on the electrode 105 surfaces, while the non-neutralized RBDs 205 will link to the hACE2 receptors 215, as shown in FIG. 2. The change in the impedance response depends on the amount of interacted RBD-hACE2 on the electrode 105 surface and, thus, a function of the NAbs' concentration. To detect NAbs, hACE2 proteins were immobilized using NHS-EDC chemistry to interact with RBDs in the sample [34]. The immobilization procedure is as follows. After rinsing the electrode surfaces with ethanol and exposing the surface to plasma for 30 s, electrodes were incubated overnight in alkanethiol solution (99% 11-mercapto-1-undecanol 1 mM and 1% 16-mercaptohexadecanoic acid 1 mM in ethanol). The activated electrodes were rinsed with ethanol and immersed for 30 minutes inside a solution of 100 mg dimethylaminopropyl carbodiimide and 40mg N-hydroxysuccinimide in 4 mL of DI water. The electrodes were then rinsed with DI water and incubated with 0.1 μg/μL hACE2 protein in PBS. Finally, electrodes were rinsed with PBS and dried. Impedance measurements of the sensors with and without hACE2 immobilization were used to ensure the protein deposition on the surface. The device sensitivity was obtained for different NAb concentrations in the buffer. The working principle is: (i) NAbs with different concentrations were mixed with SARS-CoV-2 RBD diluted in PBS. (ii) The mixture was loaded and filled into the microchamber. (iii) Sensor surfaces were previously functionalized with the hACE2, and non-neutralized RBDs interacted with the hACE2 receptors. (iv) Impedance response, which is a function of NAb concentration, was recorded.

### Results and Discussions

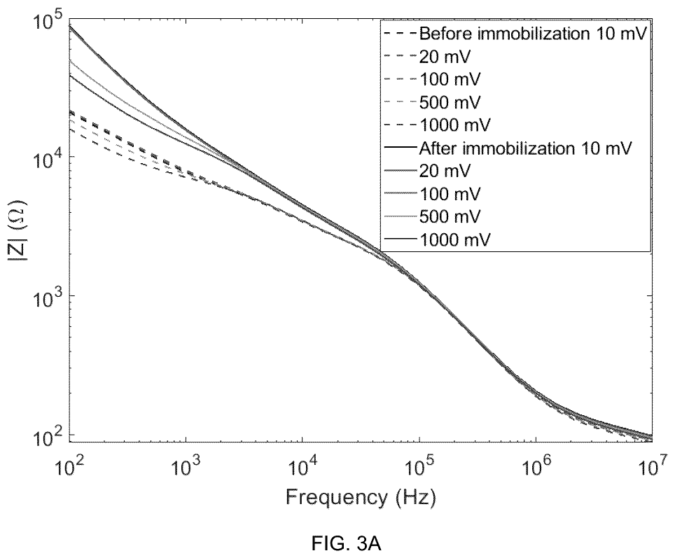

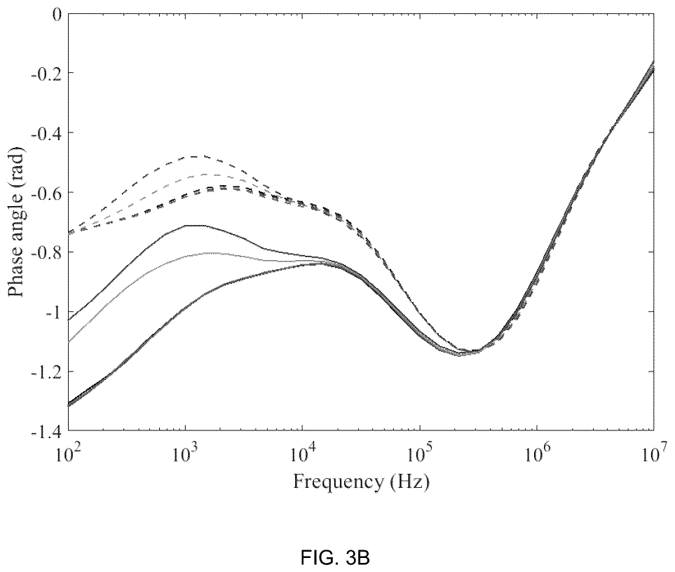

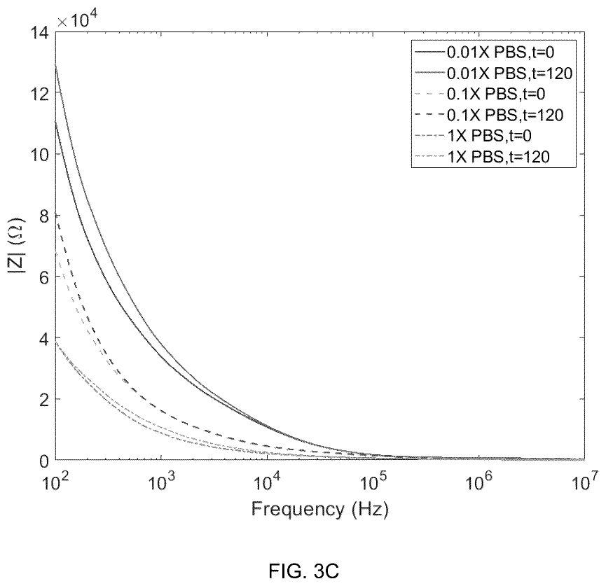

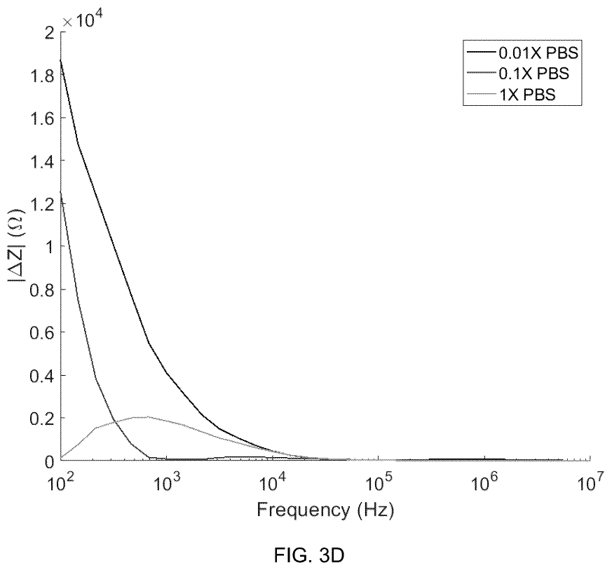

Before characterizing the biosensor detection capability, certain parameters, such as the applied voltage and frequency, needed to be determined to reach optimum operation. Also, it is important to find whether the surface has been immobilized with bovine IgG or not. Therefore, the impedances of the devices before and after immobilization were recorded at 10, 20, 100, 500, and 1000 mV in a frequency range from 100 Hz to 10 MHz to investigate these parameters. FIGS. 3A, 3B, 3C, and 3D show the impedance spectra presented in the Bode diagram to illustrate the abovementioned effects.

System impedances were measured at 10, 20, 100, 500, and 1000 mV to find the properly applied electric potential. According to FIG. 3A and FIG. 3B, increasing applied voltage amplitude has no significant effects on the impedance behavior in high frequencies from approximately 10 KHz to 10 MHz. Since in high frequencies, the impedance (Z=Rₛ+1/jωCᵢₙₜ) represented PBS resistance (nearly constant) in this frequency range. However, an increase in the applied potential (more than 100 mV) in the lower frequency range reduced the system impedance. Therefore, the applied voltage was set at 100 mV to record the impedance changes resulting from the binding, not the voltage amplitude effects.

According to FIG. 3A, at frequencies from approximately 10 kHz to 10 MHZ, the sensor impedance magnitude originated from the resistance of the electrolyte. Therefore, there was almost no change in impedance in this range even after protein immobilization because PBS resistance was nearly constant. FIG. 3B shows that in frequencies lower than ˜10 KHz, a combination of EDL capacitance and resistance dominated the sensor impedance before immobilization. However, bovine IgG covered the electrode surface after immobilization, acting as an electrical isolation layer. As a result, the impedance at low frequencies showed mostly capacitive behavior after immobilization.

To find the effective detection frequency, impedance changes for the maximum anti-bovine IgG concentration for 0.01×PBS (100 ng/ml), 0.1×PBS (100 ng/ml), and 1×PBS (10 μg/ml) were obtained from 100 Hz to 10 MHz. FIG. 3C shows the impedance of the chip as a function of frequency when the anti-bovine IgG was applied to the chamber and after 120 s. This figure demonstrates that the values for the higher PBS dilution (1×PBS) are lower due to the increase in the solution resistance. FIG. 3D shows impedance changes after 120 s for each buffer dilution. According to this figure, for 0.01×PBS and 0.1×PBS, 100 Hz is the frequency at which impedance changes are maximum. However, for 1×PBS, this frequency is about 600 Hz. Therefore, the LOD of the chip was found in these specified frequencies for other concentrations of anti-bovine IgG.

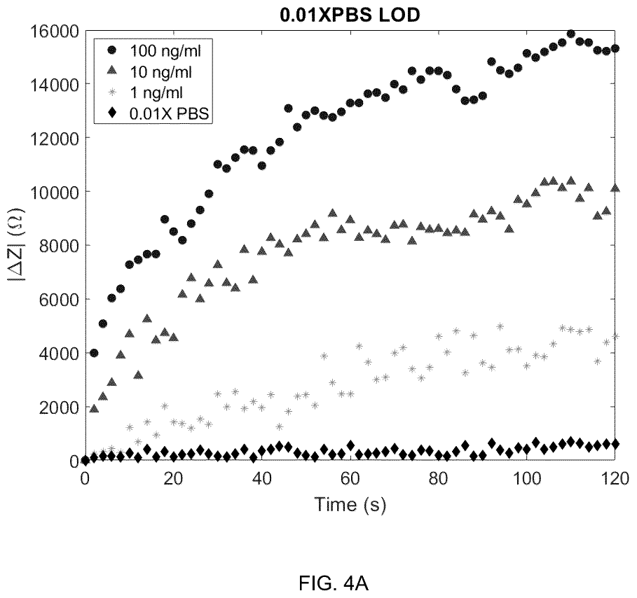

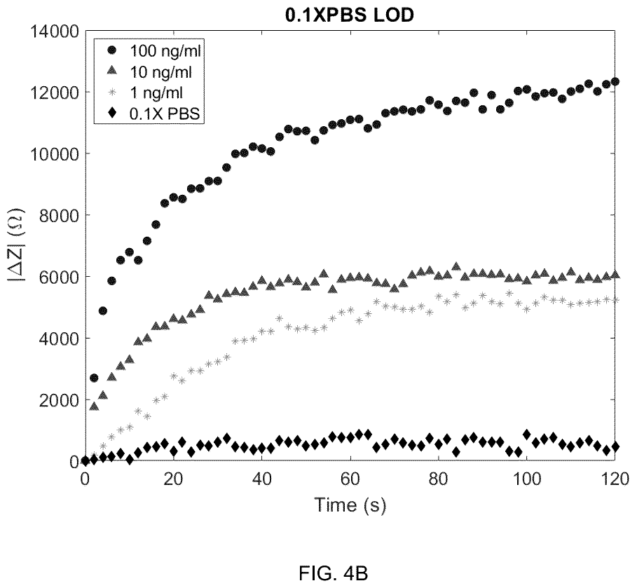

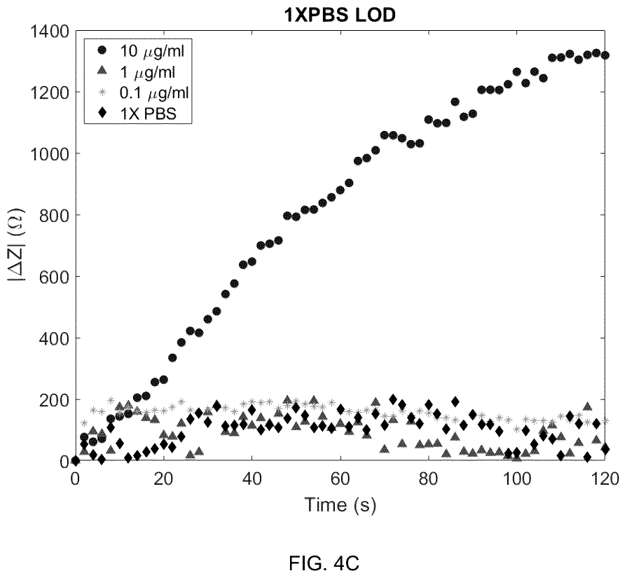

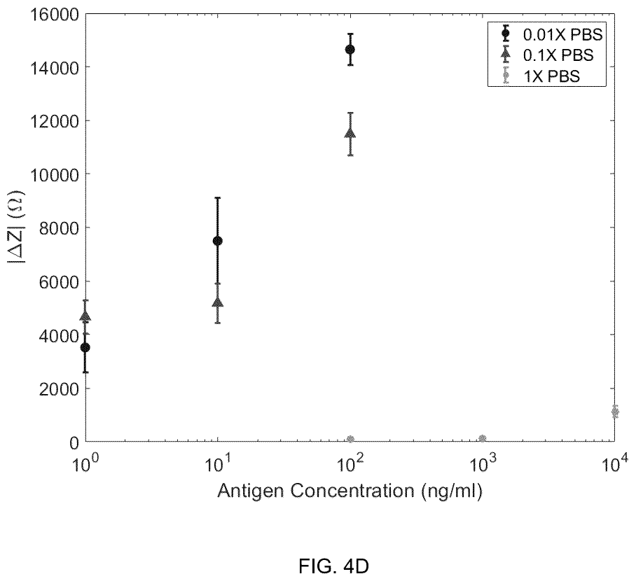

With applying proper voltage and frequency, the LOD of the device in different concentrations of PBS buffer with conductivities of ˜0.016, ˜0.16, and ˜1.6 S/m were investigated. FIG. 4A shows anti-bovine IgG detection with 1, 10, and 100 ng/ml concentrations at 100 mV and 100 Hz. After that, the same applied potential and frequency, as well as anti-bovine IgG concentrations, were used when 0.1×PBS was used to dilute bovine IgG whole molecules, as shown in FIG. 4B. FIG. 4C illustrates LOD for anti-bovine IgG. In contrast, 1×PBS (σ˜1.6 (S/m)) was used during the experiment. The anti-bovine IgG had 100 ng/ml, 1 μg/ml, and 10 μg/ml concentrations. FIG. 4D shows standard deviations (STDs) for these cases, determining LOD for all cases. Based on the Analysis of Variance (ANOVA) tests, the minimum detections for 0.01×PBS, 0.1×PBS, and 1×PBS are 1 ng/ml, 10 ng/ml, 10 μg/ml, respectively. In other words, if a blood serum sample with a conductivity close to 1×PBS were used, it would not be able to detect the antigen. However, detection will be possible when the sample is diluted by 10 or 100 times. From this aspect, the LOD of the device will be 100 ng/ml. The main reason for such a low LOD for 1×PBS is that Ad thickness is about 0.7 nm, which is smaller than IgG antibody (˜10 nm), so EDL change after the interaction is small [38]. Also, in high conductive solutions (e.g., biological samples) and low frequencies, Electrode Polarization (EP) effect can overshadow the recorded signal. Reducing this side effect can improve the LOD of interdigitated electrodes in such buffers.

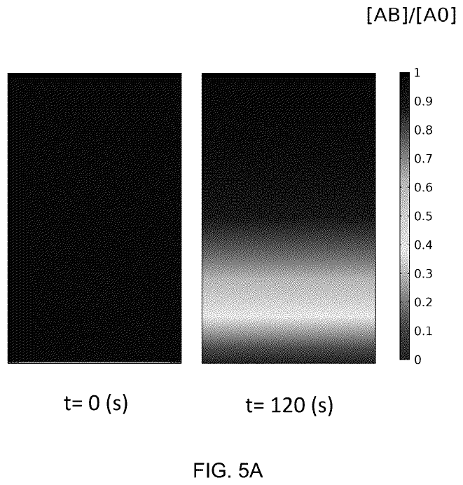

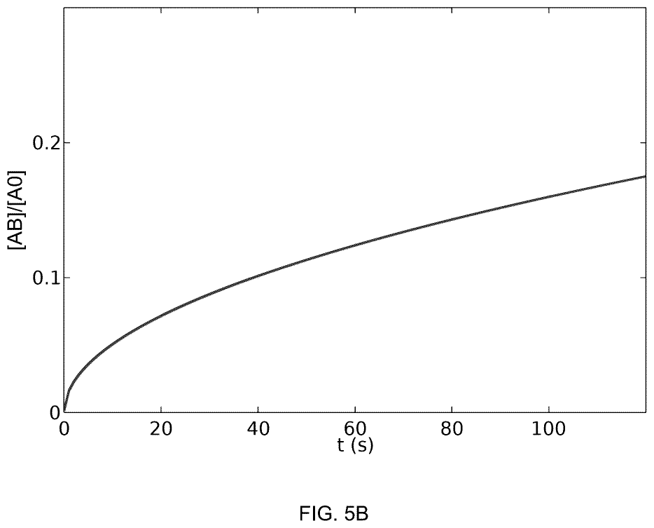

Specificity and selectivity are also necessary considerations for biosensor development. Specificity shows that the antibody only recognizes and binds to a specific antigen, whereas selectivity is the ability to differentiate the intended target molecule within a complex mixture. In order to test the specificity of the sensor IgG-free BSA was used as the control buffer. The IgG-free BSA buffer was also used to prepare the anti-bovine IgG buffers with 1 (ng/ml) and 10 (ng/ml) concentrations. FIG. 5A shows data that demonstrates no detection with the current sensor when the control buffer was applied, while there was detection in the buffer, including the anti-bovine IgG corresponding to its concentration. To determine the sensor's selectivity, mouse serum was used as the complex control buffer, and anti-bovine IgG with 1 (ng/ml) and 10 (ng/ml) were added to this buffer. FIG. 5B shows data that proves the potential of the sensor to discriminate the anti-bovine IgG from the mouse serum IgGs and shows a good target-specific response for different antigen concentrations.

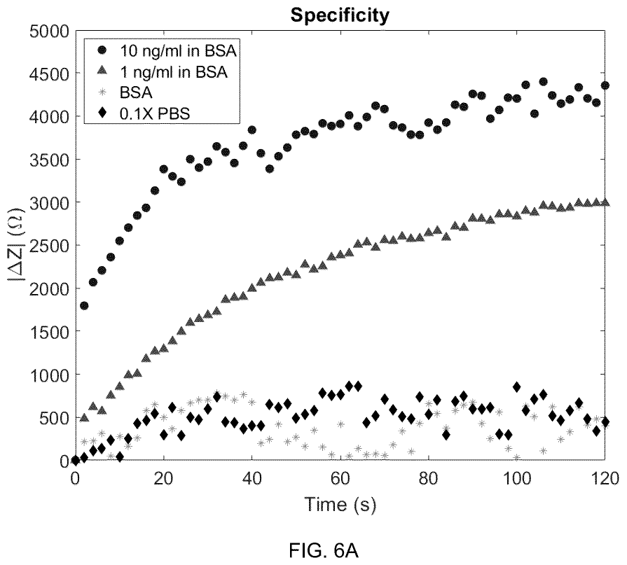

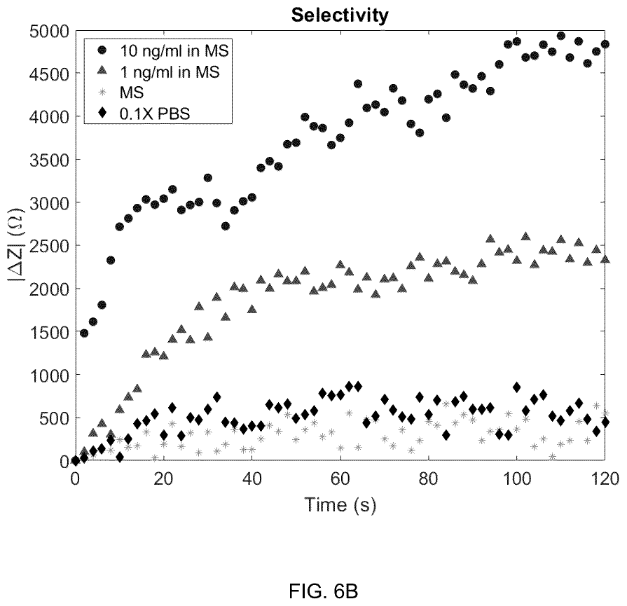

To determine the dominant transport mechanism in the device, the order of magnitudes for reagent displacement in 1 s due to the ACEK phenomena (ACEO, ACET, and DEP) and Brownian motion were obtained using Eq. 1 to Eq. 5 in the most effective voltage and frequency for each PBS dilution. According to the results shown in Table 1, Brownian motion is the dominant transport phenomenon. In other words, device detection is mainly diffusion-based instead of ACEK-based. To clarify this outcome, antibody-antigen binding has also been simulated in a pure diffusion condition, and the concentration results are shown in FIGS. 6A and 6B.

TABLE 1
Reagent displacement in the device due to the
ACEO, ACET, DEP, and Brownian motion in 1 (s).

| | 0.01XPBS | 0.1XPBS | 1XPBS |
|---|---|---|---|
| | Δx (m) in 1 sec | Δx (m) in 1 sec | Δx (m) in 1 sec |
| ACEO | 9.73 × 10⁻¹² | 3.64 × 10⁻¹⁴ | 1.12 × 10⁻¹⁶ |
| ACET | 2.22 × 10⁻¹¹ | 2.22 × 10⁻¹⁰ | 2.22 × 10⁻⁹ |
| DEP | 3.92 × 10⁻¹⁶ | 5.91 × 10⁻²² | −1.11 × 10⁻²³ |
| Brownian | 9.35 × 10⁻⁷ | 9.35 × 10⁻⁷ | 9.35 × 10⁻⁷ |
| motion | | | |

FIG. 6A shows normalized anti-bovine IgG concentration ([A]) at the initial time (t=0) and after 120 s of antibody-antigen reaction. This figure shows that antigen concentration reduces near the electrode surface due to interacting with the antibodies on the surface. FIG. 6B shows normalized concentration for the reacted protein-protein ([AB]) on the electrode surface. According to the simulation, diffusion base interaction has the same trend as the changes in impedance. The abovementioned impedance changes are mainly because of the electric isolation layer when the proteins cover the electrode surface. This layer changes interfacial electric properties such as dielectric constant. The impedance increases as the thickness of this layer increases, as shown in FIGS. 4A, 4B, 4C, and 4D.

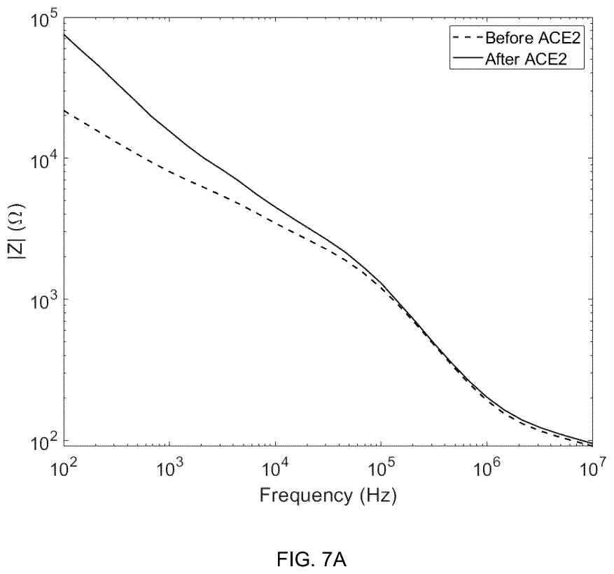

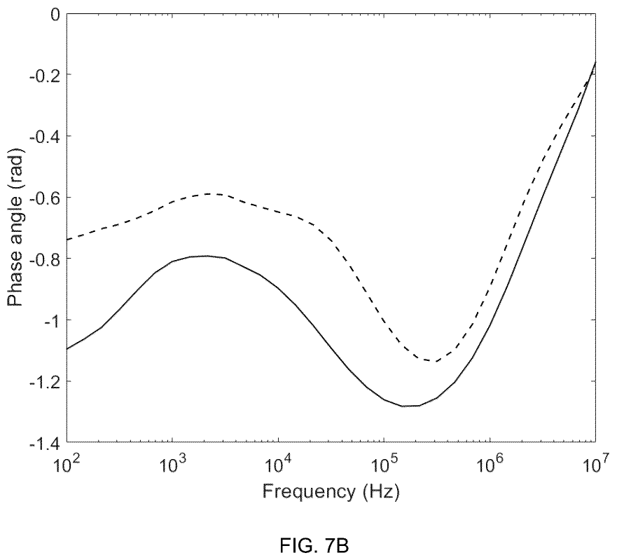

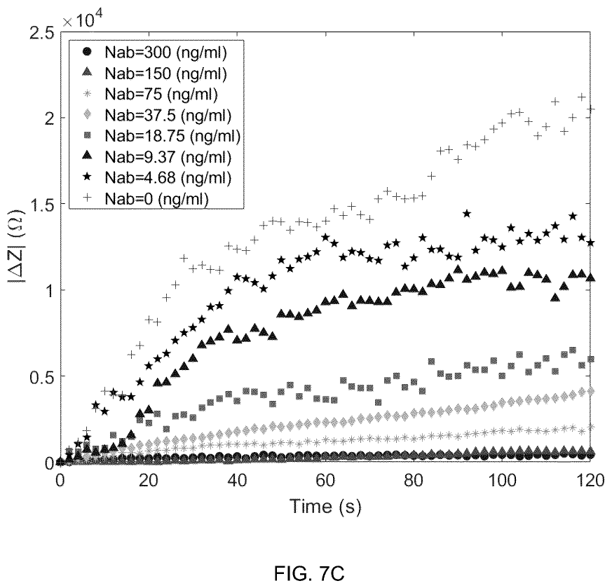

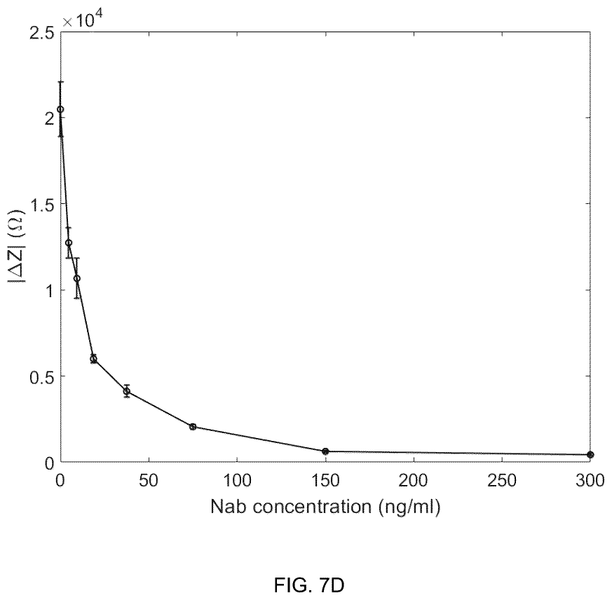

After determining the proper detection parameters, the capability of the sensor to detect SARS-CoV-2 RBD NAbs was studied. After immobilizing the electrode surface with hACE2 protein, 0.1×PBS solutions with specific NAbs concentrations were applied to the device. Changes in impedance were recorded as shown in FIGS. 7A, 7B, 7C, and 7D. FIG. 7C shows the LOD for RBD-hACE2 interaction for different NAbs concentrations. FIG. 7B shows the NAb concentration and impedance change relationship for the device. All data were obtained using 0.1×PBS buffer for dilution at 100 mV and 100 Hz with the initial impedance magnitude of 100 kΩ.

FIG. 7A shows ACE2 immobilization effects on impedance magnitude. FIG. 7B shows the phase angle of the chip impedance. FIG. 7C shows the LOD for RBD-hACE2 interaction for different NAbs concentrations. FIG. 7D shows the NAb concentration and impedance change relationship for the device. All data were obtained using 0.1×PBS buffer for dilution at 100 mV and 100 Hz. FIG. 7A shows the impedance increase at low frequencies, and FIG. 7B shows the capacitive behavior of the impedance after hACE2 immobilization on the surface. This change in the impedance is due to the electrode surface coverage by hACE2, which changes surface electrical properties.

The immobilized devices with hACE2 were connected to the impedance analyzer to record the impedance with the applied voltage of 100 mV at 100 Hz. When the SARS-CoV-2 RBD solution was introduced into the sensor, RBDs would interact with the hACE2 receptors on the surface. Due to this interaction, EDL thickness would change, which results in the impedance increase. However, NAbs prevent this interaction by deactivating the viral RBDs, as shown in FIG. 2. Therefore, depending on the NAb's concentrations in a RBD buffer, the impedance responses become different. If there were enough NAbs in the RBD solution, there would be no RBD-hACE2 interaction on the surface.

Consequently, there would be no impedance change. FIG. 7C shows data that demonstrates the results of RBD-hACE2 interaction in the presence of NAbs with different concentrations, obtained from serial dilution of the 300 ng/ml NAb solution [6]. According to this figure, when the applied NAb concentration to the device increases, there was less interaction between the RBDs and hACE2 on the electrode surface, so there was almost no change in impedance. However, when RBDs were not neutralized (such as in the case of a low concentration of NAb), more RBD and hACE2 interaction occurred on the electrode surface, resulting in greater impedance changes.

FIG. 7D shows a detection curve for this device based on the impedance changes for the known concentrations of the NAb in the RBD buffer. This curve can be used to determine the concentration of NAb in any sample since it covers the concentration range of the analytical measuring interval (AMI). In other words, a sample with an unknown NAb concentration can be introduced into the sensor, and the NAb concentration can be estimated from the resultant impedance change curve in FIG. 7D. The demonstration shows feasibility for a label-free detection with less time and cost, making it more applicable for point-of-care detection, compared to ELISA methods [6].

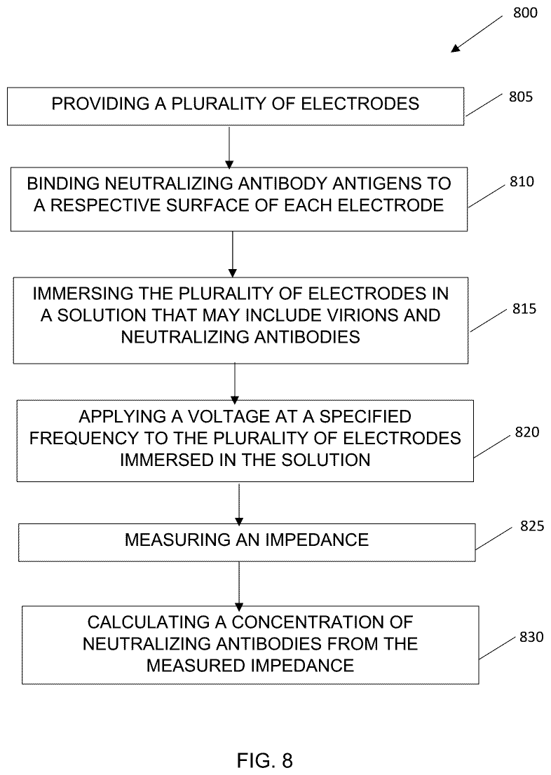

FIG. 8 shows a flowchart for a method embodiment of the present invention. Method 800 includes Block 805, providing a plurality of electrodes, and Block 810, binding neutralizing antibody antigens to a respective surface of each electrode. Method 800 further includes Block 815, immersing the plurality of electrodes in a solution that may include virions and neutralizing antibodies, and Block 820, applying a voltage at a specified frequency to the plurality of electrodes immersed in the solution. In addition, Method 800 includes Block 825, measuring an impedance, and Block 830, calculating a concentration of neutralizing antibodies from the measured impedance.

### Conclusion

In the present study, the capability of a biosensor to detect IgG protein was investigated using the impedance spectroscopy method. First, experimental parameters were determined, including applied AC voltage and frequency. The impedance spectrum for the device in a frequency range from 100 Hz to 10 MHz showed that the impedance represented buffer resistance in higher frequencies. There is no significant change in impedance in the frequency range from 10 kHz to 10 MHz. Impedance changes mostly happened in lower frequencies, and maximum change occurred at 100 Hz. Therefore, the optimum detection frequency was expected to place in the low-frequency range. To find the most effective frequency for detection, binding was studied in the same frequency range for each buffer solution in which the maximum interaction occurred at 100 Hz for 0.01×PBS and 0.1×PBS. For 1×PBS, the optimum frequency was found to be around 600 Hz. Then, the most effective applied AC magnitude was determined.

The effect of different applied AC voltages (less than 1 Vₚₚ) was recorded on the device's impedance. Results illustrated that impedance was reduced in higher applied voltages (more than 500 mV). Therefore, to eliminate this side effect on the impedance change due to the binding, a 100 mV voltage amplitude was applied to the sensor. To find out the binding efficiency of the device, anti-bovine IgG and bovine IgG interaction in three different PBS buffers of 0.01×PBS (σ˜0.016 (S/m)), 0.1×PBS (σ˜0.16 (S/m)), and 1×PBS (σ˜1.6 (S/m)) were studied. ANOVA test was used to determine LODs for different mentioned dilution buffers. The results demonstrated 1 (ng/ml), 10 (ng/ml), and 10 (μg/ml) LOD for 0.01×PBS, 0.1×PBS, and 1×PBS, respectively. Also, in previous studies, ACEK phenomena were introduced as the dominant force in such a device. Due to low electric potential magnitudes, transport in the device is dominated by Brownian motion, as shown by the scaling analysis. Therefore, detection happens mainly due to diffusion rather than ACEK transport. Numerical simulations of diffusive interactions show similar trends with the experimental data.

As an application, the present interdigitated electrode was used to detect SARS-CoV-2 neutralizing antibodies. The hACE2 receptors which interacted with RBDs were immobilized on the electrode surface. RBS buffers with different NAb concentrations were applied to the device. The results showed that in high NAb concentrations, RBDs were neutralized, and there was no interaction on the electrode surface. Therefore, impedance showed almost no change. However, in low NAb concentrations, RBDs bound to the hACE2, and impedance changes illustrated this interaction. The device has the potential to be fast, inexpensive, and easy to operate and provide a quantitative assessment of neutralizing antibodies in samples.

An embodiment of the present invention comprises, consists essentially of, or consists of a neutralizing antibody detection system comprising: a plurality of electrodes, wherein neutralizing antibody antigens are bound to a respective surface of each electrode, and wherein the plurality of electrodes with the neutralizing antibody antigens are configured to bind virions when immersed in a solution containing the virions; and an electrical circuit connected to the plurality of electrodes to apply a voltage at a specified frequency and to measure a respective impedance. In one aspect, the plurality of electrodes includes interdigitated electrodes. In another aspect, the plurality of electrodes includes a layer of conductive material. In another aspect, the virions are SARS-CoV-2 virions and the neutralizing antibody antigens are SARA-CoV-2 neutralizing antibody antigens. In another aspect, the specified frequency is greater than or equal to 10 Hz and less than or equal to 1000 Hz. In another aspect, the specified frequency is greater than or equal to 100 Hz and less than or equal to 1000 Hz. In another aspect, the specified frequency is greater than or equal to 100 Hz and less than or equal to 600 Hz. In another aspect, the specified frequency is 100 Hz, 200 Hz, 300 Hz, 400 Hz, 500 Hz, 600 Hz, 700 Hz, 800 Hz, 900 Hz, or 1000 Hz.

Another embodiment of the present invention comprises, consists essentially of, or consists of a method of detecting neutralizing antibodies comprising providing a plurality of electrodes; providing a plurality of electrodes; binding neutralizing antibody antigens to a respective surface of each electrode; immersing the plurality of electrodes in a solution that may include virions and neutralizing antibodies; applying a voltage at a specified frequency to the plurality of electrodes immersed in the solution; measuring an impedance; and calculating a concentration of neutralizing antibodies from the measured impedance. In one aspect, the plurality of electrodes includes interdigitated electrodes. In another aspect, the plurality of electrodes includes a layer of conductive material. In another aspect, the neutralizing antibody antigens are human angiotensin-converting enzyme 2 receptors. In another aspect, the virions are SARS-CoV-2 virions and the neutralizing antibody antigens are SARA-CoV-2 neutralizing antibody antigens. In another aspect, the specified frequency is greater than or equal to 10 Hz and less than or equal to 1000 Hz. In another aspect, the specified frequency is greater than or equal to 100 Hz and less than or equal to 1000 Hz. In another aspect, the specified frequency is greater than or equal to 100 Hz and less than or equal to 600 Hz. In another aspect, the specified frequency is 100 Hz, 200 Hz, 300 Hz, 400 Hz, 500 Hz, 600 Hz, 700 Hz, 800 Hz, 900 Hz, or 1000 Hz.

It is contemplated that any embodiment discussed in this specification can be implemented with respect to any method, kit, apparatus or system of the invention, and vice versa.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

[1] C.B. Jackson, M. Farzan, B. Chen, H. Choc, Mechanisms of SARS-CoV-2 entry into cells, Nature reviews Molecular cell biology, 23 (2022) 3-20.
[2] S.C. Taylor, B. Hurst, C.L. Charlton, A. Bailey, J.N. Kanji, M.K. McCarthy, T.E. Morrison, L. Hucy, K. Annen, M.G. DomBourian, A New SARS-CoV-2 Dual-Purpose Serology Test: Highly Accurate Infection Tracing and Neutralizing Antibody Response Detection, Journal of Clinical Microbiology, 59 (2021) e02438-02420.
[3] D. Cromer, M. Steain, A. Reynaldi, T.E. Schlub, A.K. Wheatley, J.A. Juno, S.J. Kent, J.A. Triccas, D.S. Khoury, M.P. Davenport, Neutralising antibody titres as predictors of protection against SARS-CoV-2 variants and the impact of boosting: a meta-analysis, The Lancet Microbe, (2021).
[4] M.A. Black, G. Shen, X. Feng, W.F.G. Beltran, Y. Feng, V. Vasudevaraja, D. Allison, L.H. Lin, T. Gindin, M. Astudillo, Analytical performance of lateral flow immunoassay for SARS-CoV-2 exposure screening on venous and capillary blood samples, Journal of immunological methods, 489 (2021) 112909.
[5] J. Nie, Q. Li, J. Wu, C. Zhao, H. Hao, H. Liu, L. Zhang, L. Nie, H. Qin, M. Wang, Establishment and validation of a pseudovirus neutralization assay for SARS-CoV-2, Emerging microbes & infections, 9 (2020) 680-686.
[6] Food, D. Administration, USA. cPass™ SARS-CoV-2 neutralization antibody detection kit “https://www.genscript.com/covid-19-detection-fda-cua.html”.
[7] P. Kongsuphol, H. Jia, H.L. Cheng, Y. Gu, B.D. Shunmuganathan, M.W. Chen, S.M. Lim, S.Y. Ng, P.A. Tambyah, H. Nasir, A rapid simple point-of-care assay for the detection of SARS-CoV-2 neutralizing antibodies, Communications medicine, 1 (2021) 1-12.
[8] S.M. Lim, H.L. Cheng, H. Jia, P. Kongsuphol, B. D/O Shunmuganathan, M.W. Chen, S.Y. Ng, X. Gao, S.P. Turaga, S.P. Heussler, Finger stick blood test to assess postvaccination SARS-CoV-2 neutralizing antibody response against variants, Bioengineering & Translational Medicine, (2022) e10293.
[9] L. Davidi, S. Moraïs, L. Artzi, D. Knop, Y. Hadar, Y. Arfi, E.A. Bayer, Toward combined delignification and saccharification of wheat straw by a laccase-containing designer cellulosome, Proceedings of the National Academy of Sciences, 113 (2016) 10854-10859.
[10] Y. Huang, J. Xu, J. Liu, X. Wang, B. Chen, Disease-related detection with electrochemical biosensors: a review, Sensors, 17 (2017) 2375.
[11] N. Sandhyarani, Surface modification methods for electrochemical biosensors, electrochemical Biosensors, Elsevier2019, pp. 45-75.
[12] A.M. Faria, E.B. Peixoto, C.B. Adamo, A. Flacker, E. Longo, T. Mazon, Controlling parameters and characteristics of electrochemical biosensors for enhanced detection of 8-hydroxy-2′-deoxyguanosine, Scientific reports, 9 (2019) 1-10.
[13] E.E. Ferapontova, Hybridization biosensors relying on electrical properties of nucleic acids, Electroanalysis, 29 (2017) 6-13.
[14] J. Shi, D.M. Porterfield, Surface modification approaches for electrochemical biosensors, Biosensors-Emerging Materials and Applications, (2011).
[15] S.S. Mahshid, S.E. Flynn, S. Mahshid, The potential application of electrochemical biosensors in the COVID-19 pandemic: A perspective on the rapid diagnostics of SARS-CoV-2, Biosensors and Bioelectronics, 176 (2021) 112905.
[16] N.J. Ronkainen, H.B. Halsall, W.R. Heineman, Electrochemical biosensors, Chemical Society Reviews, 39 (2010) 1747-1763.
[17] H. Cui, S. Li, Q. Yuan, A. Wadhwa, S. Eda, M. Chambers, R. Ashford, H. Jiang, J. Wu, An AC electrokinetic impedance immunosensor for rapid detection of tuberculosis, Analyst, 138 (2013) 7188-7196.
[18] S. Li, H. Cui, Q. Yuan, J. Wu, A. Wadhwa, S. Eda, H. Jiang, AC electrokinetics-enhanced capacitive immunosensor for point-of-care serodiagnosis of infectious diseases, Biosensors and Bioelectronics, 51 (2014) 437-443.
[19] S. Li, Y. Ren, H. Jiang, Convection and mass transfer enhanced rapid capacitive serum immunoassay, Rsc Advances, 4 (2014) 9064-9071.
[20] C. Cheng, S. Wang, J. Wu, Y. Yu, R. Li, S. Eda, J. Chen, G. Feng, B. Lawrie, A. Hu, Bisphenol a sensors on polyimide fabricated by laser direct writing for onsite river water monitoring at attomolar concentration, ACS applied materials & interfaces, 8 (2016) 17784-17792.
[21] H. Cui, C. Cheng, X. Lin, J. Wu, J. Chen, S. Eda, Q. Yuan, Rapid and sensitive detection of small biomolecule by capacitive sensing and low field AC electrothermal effect, Sensors and Actuators B: Chemical, 226 (2016) 245-253.
[22] A. Mansoorifar, A. Koklu, A.C. Sabuncu, A. Beskok, Dielectrophoresis assisted loading and unloading of microwells for impedance spectroscopy, Electrophoresis, 38 (2017) 1466-1474.
[23] S. Park, M. Koklu, A. BeskoK, Particle trapping in high-conductivity media with electrothermally enhanced negative dielectrophoresis, Analytical chemistry, 81 (2009) 2303-2310.
[24] S. Park, A. Beskok, Alternating current electrokinetic motion of colloidal particles on interdigitated microelectrodes, Analytical Chemistry, 80 (2008) 2832-2841.
[25] A. Castellanos, A. Ramos, A. Gonzalez, N.G. Green, H. Morgan, Electrohydrodynamics and dielectrophoresis in microsystems: scaling laws, Journal of Physics D: Applied Physics, 36 (2003) 2584.
[26] C.-H. Chu, I. Sarangadharan, A. Regmi, Y.-W. Chen, C.-P. Hsu, W.-H. Chang, G.-Y. Lee, J.-I. Chyi, C.-C. Chen, S.-C. Shiesh, Beyond the Debye length in high ionic strength solution: direct protein detection with field-effect transistors (FETs) in human serum, Scientific reports, 7 (2017) 1-15.
[27] A. Koklu, A. El Helou, P.E. Raad, A. Beskok, Characterization of temperature rise in alternating current electrothermal flow using thermoreflectance method, Analytical chemistry, 91 (2019) 12492-12500.
[28] A. Beskok, A. Koklu, J. Chiao, Sensor and Method for Detecting Target Molecules, Google Patents, 2021.
[29] S. Bakhtiari, M.K. Manshadi, A. Mansoorifar, A. Beskok, A Microfluidic Dielectric Spectroscopy System for Characterization of Biological Cells in Physiological Media, Sensors, 22 (2022) 463.
[30] A. Mansoorifar, A. Koklu, S. Ma, G.V. Raj, A. Beskok, Electrical impedance measurements of biological cells in response to external stimuli, Analytical chemistry, 90 (2018) 4320-4327.
[31] N. Sinha, S. Mohan, C.A. Lipschultz, S.J. Smith-Gill, Differences in electrostatic properties at antibody-antigen binding sites: implications for specificity and cross-reactivity, Biophysical journal, 83 (2002) 2946-2968.
[32] E. Rosenberg, Aggregation of therapeutic antibodies in the course of downstream processing, lmu, 2010.
[33] C.-K. Yang, J.-S. Chang, S.D. Chao, K.-C. Wu, Effects of diffusion boundary layer on reaction kinetics of immunoassay in a biosensor, Journal of Applied Physics, 103 (2008) 084702.
[34] J. Yang, S.J. Petitjean, M. Koehler, Q. Zhang, A.C. Dumitru, W. Chen, S. Derclaye, S.P. Vincent, P. Soumillion, D. Alsteens, Molecular interaction and inhibition of SARS-CoV-2 binding to the ACE2 receptor, Nature communications, 11 (2020) 1-10.

What is claimed is:

1. A neutralizing antibody detection system comprising:
a plurality of electrodes, wherein neutralizing antibody antigens are bound to a respective surface of each electrode, and wherein the plurality of electrodes with the neutralizing antibody antigens are configured to bind virions when immersed in a solution containing the virions; and
an electrical circuit connected to the plurality of electrodes, to apply a voltage at a specified frequency and to measure a respective impedance.

2. The system of claim 1, wherein the plurality of electrodes comprises interdigitated electrodes.

3. The system of claim 1, wherein the plurality of electrodes comprises a layer of conductive material.

4. The system of claim 1, wherein the neutralizing antibody antigens are human angiotensin-converting enzyme 2 receptors.

5. The system of claim 1, wherein the virions are SARS-CoV-2 virions and the neutralizing antibody antigens are SARA-CoV-2 neutralizing antibody antigens.

6. The system of claim 1, wherein the specified frequency is greater than or equal to 10 Hz and less than or equal to 1000 Hz.

7. The system of claim 6, wherein the specified frequency is greater than or equal to 100 Hz and less than or equal to 1000 Hz.

8. The system of claim 6, wherein the specified frequency is greater than or equal to 100 Hz and less than or equal to 600 Hz.

9. The system of claim 6, wherein the specified frequency is 100 Hz, 200 Hz, 300 Hz, 400 Hz, 500 Hz, 600 Hz, 700 Hz, 800 Hz, 900 Hz, or 1000 Hz.

10. A method of detecting neutralizing antibodies comprising:
providing a plurality of electrodes;
binding neutralizing antibody antigens to a respective surface of each electrode;
immersing the plurality of electrodes in a solution that may include virions and neutralizing antibodies;
applying a voltage at a specified frequency to the plurality of electrodes immersed in the solution;
measuring an impedance; and
calculating a concentration of neutralizing antibodies from the measured impedance.

11. The method of claim 10, wherein the plurality of electrodes comprises interdigitated electrodes.

12. The method of claim 10, wherein the plurality of electrodes comprises a layer of conductive material.

13. The method of claim 10, wherein the neutralizing antibody antigens are human angiotensin-converting enzyme 2 receptors.

14. The method of claim 10, wherein the virions are SARS-CoV-2 virions and the neutralizing antibody antigens are SARA-CoV-2 neutralizing antibody antigens.

15. The method of claim 10, wherein the specified frequency is greater than or equal to 10 Hz and less than or equal to 1000 Hz.

16. The method of claim 15, wherein the specified frequency is greater than or equal to 100 Hz and less than or equal to 1000 Hz.

17. The method of claim 15, wherein the specified frequency is greater than or equal to 100 Hz and less than or equal to 600 Hz.

18. The method of claim 15, wherein the specified frequency is 100 Hz, 200 Hz, 300 Hz, 400 Hz, 500 Hz, 600 Hz, 700 Hz, 800 Hz, 900 Hz, or 1000 Hz.
